(19) United States
(12) Patent Application Publication
(10) Pub. No.: US 2002/0028763 A1
(43) Pub. Date: Mar. 7, 2002

(54) Use of ADNF for the treatment of retinal or optic nerve head damage

(76) Inventors: Debra L. Shade, Benbrook, TX (US); Abbot F. Clark, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 09/920,931

(22) Filed: Aug. 2, 2001

Related U.S. Application Data
(60) Provisional application No. 60/230,815, filed on Sep. 7, 2000.

(51) Int. Cl. A61K 38/17

(52) U.S. Cl. 514/2

(57) ABSTRACT

Methods for treating retinal and/or optic nerve head damage using Activity Dependent Neurotrophic Factor are disclosed.

This application claims priority from U.S. Provisional Application Ser. No. 60/230,815 filed Sep. 7, 2000.

The present application is directed to the use of Activity Dependent Neurotrophic Factor (ADNF) for the treatment of retinal and/or optic nerve head damage.

### BACKGROUND OF THE INVENTION

The glaucomas are a heterogeneous group of optic neuropathies characterized by the cupping of the optic nerve head, thinning of the retinal nerve fiber layer due to loss of retinal ganglion cells, and specific pathogenetic changes in the visual field. Although elevated intraocular pressure (IOP) is an important risk factor for the development of many common forms of glaucoma (Sommer, A. et al., Arch. Ophthalmol., 109:1090-1095 (1991), the phenomenon of normal tension glaucoma has been clinically established in ophthalmology (Flammer, J., Fortschr. Ophthalmol. 87:187(1990). Normal tension glaucoma is characterized by an intraocular pressure which is in the normal range, i.e., not increased, but in which the optic nerve disk is pathologically excavated and the field of vision is impaired.

At the present time glaucoma, including normal tension glaucoma, is treated by medically and/or surgically lowering elevated pressure; however, even when IOP is maintained with in a normal range visual field loss may progress. Degeneration involving retinal ganglion cells may be related to compression of the nerve fiber bundles, excitotoxicity, ischemia, or other as yet unrecognized causitive factors. Thus, factors other than IOP may play a role in determining both the occurrence and rate of progression of retinal ganglion cell death and subsequent visual field loss.

Using laboratory models, including ischemia, optic nerve crush, optic nerve transection, and cultured retinal ganglion cells (Adachi, K. et al., Eur. J. Pharmacol., 350:53-57, (1998); Yoles, E. et al., Arch. Opthalmol., 116:906-910, (1998); Di Polo, A. et al., Proc. Natl. Acad. Sci, USA 95:3978-83, (1998); Caprioli, J. et al., Invest. Ophthalmol. Vis. Sci., 37:2376-2381, (1996); Woldemussie, E. et al., Invest. Ophthalmol. Vis. Sci., 38:S100, (1997)), various pharmacological agents have been tested as potential neuroprotective approaches designed to reduce retinal ganglion cell loss. These approaches have suggested that antagonism of excitotoxicity or supplementation of neurotrophic factors can protect retinal ganglion cells from degeneration in animal models. The use of compounds capable of reducing glutamate toxicity (WO 94/13275) and polyamine antagonists (U.S. Pat. No. 5,710,165) to protect retinal ganglion cells and reduce visual field loss associated with glaucoma have been disclosed. The protective effect of MK-801, a glutamate antagonist, in a rat model of ocular hypertension, was reported. (P. Chaudhary et al., Brain Research, Vol. 792:154-158, 1998).

ADNF is a glia-derived protein which has been found to be neuroprotective at femtomolar concentrations. ADNF is both a regulator of activity dependent neuronal survival and a neuroprotectant, Gozes, et al., Developmental Brain Research, Vol. 99(2):167-175, Apr. 18, 1997; Brenneman, et al., JPET, Vol. 285:619-627, 1998; and WO 96/11948). Gozes, et al., also describe ADNF as protective against a broad range of toxins relative to Alzheimer's disease, human immunodeficiency virus (HIV), excitotoxicity, and electrical blockade. They propose the compound for development against neurodegeneration. Gozes, et al., Journal Molecular Neuro Science, Vol. 7(4):235-244, 1996, Winter). U.S. Pat. No. 5,767,240 discloses that ADNF protein increases survival of activity dependent spinal cord nerves and cerebral cortical nerves, and prevents neuronal cell death resulting from HIV. A recent publication by Guo, et al., Proc. Natl. Acad. Sci, Vol. 96:4125-4130, March, 1999, discloses that certain neurotrophic factors, including ADNF, protect hippocampal neurons which contain presenilin-1 mutations from glutamate induced cytotoxicity. WO 98/35042 discloses the use of ADNF III for conditions leading to neuronal cell death. ADNF III, previously known as ADNP, is a separate gene from ADNF, (Gozes, et al., J. of Molecular Neuroscience, Vol. 14:61-68, 2000). None of these references disclose or suggest the use of ADNF and related compounds for use in treating glaucoma.

### SUMMARY OF THE INVENTION

This invention is directed to treating retinal and/or optic nerve head damage with ADNF.

### DETAILED DESCRIPTION OF PREFERRED EMBODIMENTS

ADNF is believed to be useful to treat retinal and/or optic nerve head damage. As used herein ADNF refers to ADNF, ADNF peptides (including ADNF-9, ADNF 14), ADNF peptidimemetics, ANDF small molecule analogues, and any agent that upregulates endogenous ADNF, or an expression vector which induces ADNF expression (Compounds). Retinal and/or optic nerve head damage can result from, e.g., glaucomatous optic neuropathy, ischemic optic neuropathies, ischemic retinal diseases, degenerative retinal diseases, retinal vein occlusion, retinal artery occlusion, diabetic retinopathy, and age-related macular degeneration (ARMD).

The Compounds can be administered in a variety of ways to achieve therapeutic concentrations at the retina and/or optic nerve head. For example, the Compounds can be administered topically, by ocular injection, including, intraocular is or periocular injection, implantation of a slow release device, bolus, or encapsulated cells which will secrete Compound.

We claim:

1. A method for treating retinal and/or optic nerve head damage which comprises administering a pharmaceutically effective amount of Activity Dependent Neurotrophic Factor (ADNF).
